(19) United States
(12) Patent Application Publication
(10) Pub. No.: US 2006/0089540 A1
(43) Pub. Date: Apr. 27, 2006

(54) Device for diabetes management

(76) Inventor: Alan Meissner, Chattaroy, WA (US)

Correspondence Address: Zilka-Kotab, PC, P.O. BOX 721120, SAN JOSE, CA, 95172-1120, US

(21) Appl. No.: 10/975,176

(22) Filed: Oct. 27, 2004

(51) Int. Cl. A61B 5/00

(52) U.S. Cl. 600/300; 128/921

(57) ABSTRACT

A device for assisting in managing food and medicine intake includes a plurality of alarms each set to correspond to a time for a patient to intake at least one of food and medicine; a display for displaying a time setting for each of the alarms; a mechanism for storing verbal messages containing recorded human voice instructions for taking the at least one of the food and medicine; and a mechanism for audibly outputting the verbal messages.

### FIELD OF THE INVENTION

The present invention relates to a device for managing food and medicine intake, and more particularly, this invention relates to a device having both alarm and human voice recording capabilities.

### BACKGROUND OF THE INVENTION

Diabetes describes disease afflicting an estimated sixteen million Americans, and 135 million people world-wide. The disease is characterized by high levels of blood glucose resulting from an inability to produce a sufficient amount or properly use insulin, a hormone necessary for the body to properly convert sugar, starches and other food into a cellular energy source. There are four types of diabetes that are recognized, including: type 1 Insulin Dependent Diabetes Mellitus (IDDM or juvenile diabetes); type 2 Non-Insulin Dependent Diabetes Mellitus (NIDDM or adult onset diabetes); gestational diabetes; and diabetes caused by surgery, drugs, malnutrition, infections, and illnesses.

Complications from diabetes are severe, disabling, and may potentially lead to death. Among the complications are heart disease and stroke, which is the leading cause of diabetes-related deaths, high blood pressure, blindness, kidney disease, nervous system damage, amputations, dental disease, pregnancy complications, diabetic ketoacidosis, and hypersmolar nonketotoic coma. Each of these complications is greatly and adversely influenced by the presence of diabetes. For example, heart disease death rates and risk of stroke are two to four times higher in adults with diabetes than in adults without diabetes. Diabetes is the leading cause of new cases of blindness in adults twenty to seventy-four years in age, and is also the leading cause of end-stage renal disease.

While there is no known cure for diabetes, treatment addresses management of blood glucose levels through dietary control, exercise and medication. The treatment requires a strict regimen that may include of a combination of insulin injections and/or oral medication, multiple daily blood glucose tests, a carefully calculated diet, and planned physical activity. In spite of awareness of the need to follow the regimen, and the seriousness of the consequences of failure to follow it, many diabetics find management to be very difficult at best. In one study of more than 600 diabetes patients, two-thirds agreed that following doctor's recommendations for diabetes care is not easy, and 41 percent indicated that they did not feel successful at managing their diabetes. The largest problem areas identified were related to exercise and diet, which are critical aspects of diabetes care.

According to clinical studies sponsored by the National Institutes of Health, better management would enable people with diabetes to reduce or significantly delay the onset of many of the serious diabetes-related health complications. Nevertheless, and as aforementioned, the management of diabetes has been particularly difficult in those afflicted with the disease. As a result, the annual financial toll has been estimated by the American Diabetes Association at more than $92 billion dollars in the United States in medical expenses, disability and lost wages each year.

Efforts at improving self-management are being made, with significant direct expenditures coming from the United States Congress. In the 1997 Balanced Budget Act, coverage for self-management services provided outside of hospital settings were estimated at a five year amount of $2.1 billion. The goal was to help Medicare beneficiaries better control their blood sugar levels and reduce expensive complication of diabetes. An additional $150 million over five years was targeted to research on type 1 diabetes, with the same amount directed to Indian Health Services for more intensive diabetes prevention and treatment services. Discretionary Health and Human Services spending was set to $345 million for fiscal year 1997 alone. Nevertheless, and in spite of these enormous expenditures, there still remains a serious and unfilled need for a way to provide effective self-management.

There have been a number of devices, systems and methodologies introduced in the prior art. These references and their teachings, as they may pertain to the design and implementation of the present invention, are incorporated herewith. While specific features are discussed hereinbelow, it will be evident that these documents would in various sections not specifically referenced provide a base upon which those of ordinary skill in the art, given the present disclosure, could be directed and guided to implement the present invention, without further undue effort or research. Each of these relevant sections, referenced or otherwise, are considered to be incorporated herein for the purposes of enabling those skilled in the art to make and use the present invention.

In regard to diabetes management systems, U.S. Pat. No. 4,731,726 to Allen, III discloses a checkbook-size portable monitor and management device that includes components for measuring, storing and providing blood glucose values. In addition to the monitoring of blood glucose, a dietary assessment module is provided. Food intake, caloric intake and food exchange are listed as the type of assessment. In U.S. Pat. No. 5,019,974 to Beckers, a hand-held diabetes management device includes features of recording information relating to insulin types and doses, diet, exercise, and other data. Insulin therapy is described, including an audible alarm at a programmed time. Diet therapy is described using one of an exchange system, above/normal/below, or followed/skipped system. A low battery symbol is also provided. There is a brief description of the software, and a reference to the program stored in a 32 KB memory chip. U.S. Pat. No. 5,107,469 to Dodson describes portable reflectance photometer systems having an LCD display and also being able to sound alarm signals to alert diabetics of the need for monitoring glucose levels, receiving an insulin injection, or ingesting requisite food supplements. The device of U.S. Pat. No. 5,307,263 to Brown is primarily directed to children afflicted with diabetes. A Nintendo of America “Game Boy” serves as the hand-held computer, and cartridges are provided to control the device and performed desired functions. Other devices are described as alternatives to the Nintendo Game Boy, including palm top computers and personal appointment calendars. Therein, they state that they provide a self-care blood glucose monitoring system which adapts a hand held microprocessor unit for supplying control signals and signals representative of food intake or other useful information and for displaying information or instruction from a health care professional.

These prior art diabetes management systems fail to provide a convenient way for the entire range of diabetic population to readily manage their care. In particular, these devices are not designed to be body attached nor do they have the simple functioning required by juvenile diabetics and also functioning preferred by older diabetics.

In the prior art, there are also watches provided with various physiological monitoring. While these are not generally a part of the present-day diabetes management approach, the teachings incorporated herein by reference to these devices are important to the understandings of the operation and functioning of the present invention, and an appreciation for the novelty therein. In U.S. Pat. No. 4,837,719 to McIntosh et al, a temperature monitor, blood pressure monitor, and pulse rate monitor are combined together with a timer/alarm. In U.S. Pat. No. 5,012,229 to Lennon et al, a wrist watch includes basic heart rate monitoring in conjunction with time keeping and Med-Alert type data. In U.S. Pat. No. 5,602,802 to Leigh-Spencer et al, a portable medication reminder is disclosed as being provided with a hole to facilitate attachment of the module to a separate article regularly carried by the patient, for example, a key ring. A low-battery indication is also provided. In U.S. Pat. No. 5,628,324 to Sarbach a bio-monitoring device may be placed on the handlebar of a bicycle, in the manner most suited to the type of use, and may be combined with time or stopwatch functions. Display means comprise a conventional hour, minute and second display, and a special display of the parameters measured by the processing device. The display may also comprise flags and symbols corresponding to parameters to be displayed or to certain special circumstances, for example the end of the batteries' life. The watch also comprises push buttons enabling different functions to be called. In U.S. Pat. No. 5,691,932 to Reiner et al, a care-giver data collection and reminder system includes schedules to remind the care giver of medication times, feedings, etc. Icons are provided to assist in international use and to simplify the operation of the device. Alarms can be initiated by the micro-controller and can either be a display device or auditory. The apparatus includes a carrying case that allow the operator to attach the device on a changing table, to their belt, refrigerator, or to carry it around the neck or wrist. In U.S. Pat. No. 5,719,780 to Holmes and Moe, a medication reminder takes a wrist watch configuration, and also includes a low battery indicator. The device may be placed in a pill box, or may be worn around the neck or provided on a key chain.

In the area of diet management, U.S. Pat. No. 5,542,420 to Goldman et al discloses a diet management system implemented in a notebook PC or similar device. Included with the diet management is the requirement for pharmaceuticals and carbohydrates. U.S. Pat. No. 4,911,256 by Attikiouzel and U.S. Pat. No. 5,233,520 by Kretsch et al each disclose “smart scales” that are programmed to help with dietary management, and U.S. Pat. No. 5,691,927 to Gump discloses a hand-held food pyramid diet calculator.

Additional patents that are incorporated herein for their more general teachings of related programming and hardware and software implementation are U.S. Pat. No. 4,858,207 by Buchner, U.S. Pat. No. 5,233,571 by Wirtschafter, U.S. Pat. No. 5,371,687 by Holmes, II et al, U.S. Pat. No. 5,678,571 by Brown, U.S. Pat. No. 5,701,894 to Cherry et al, and U.S. Pat. No. Des. 390,666 to Lagerloff.

### SUMMARY OF THE INVENTION

The present invention provides a device for assisting a person having a medical condition and/or a caregiver to manage food and medicine intake. The device is designed to be simple enough for young children to use, yet provide the essential functionality required to effectively assist in management of the medical condition. The device is particularly applicable to management of medical conditions such as diabetes requiring precise timing of food and/or medicine intake.

Other aspects and advantages of the present invention will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, as well as the preferred mode of use, reference should be made to the following detailed description read in conjunction with the accompanying drawings.

FIG. 1 is a basic system diagram of the device according to one embodiment.

FIG. 2 is a perspective view of the device according to one embodiment.

FIG. 3 is a perspective view of the device according to another embodiment.

FIG. 4 is a perspective view of the device according to yet another embodiment.

### BEST MODE FOR CARRYING OUT THE INVENTION

The following description is the best embodiment presently contemplated for carrying out the present invention. This description is made for the purpose of illustrating the general principles of the present invention and is not meant to limit the inventive concepts claimed herein.

The present invention provides a device for assisting a person having a medical condition and/or a caregiver to manage food and medicine intake. The device is designed to be simple enough for young children to use, yet provide the essential functionality required to effectively assist in management of the medical condition. The device is particularly applicable to management of medical conditions such as diabetes requiring precise timing of food and/or medicine intake.

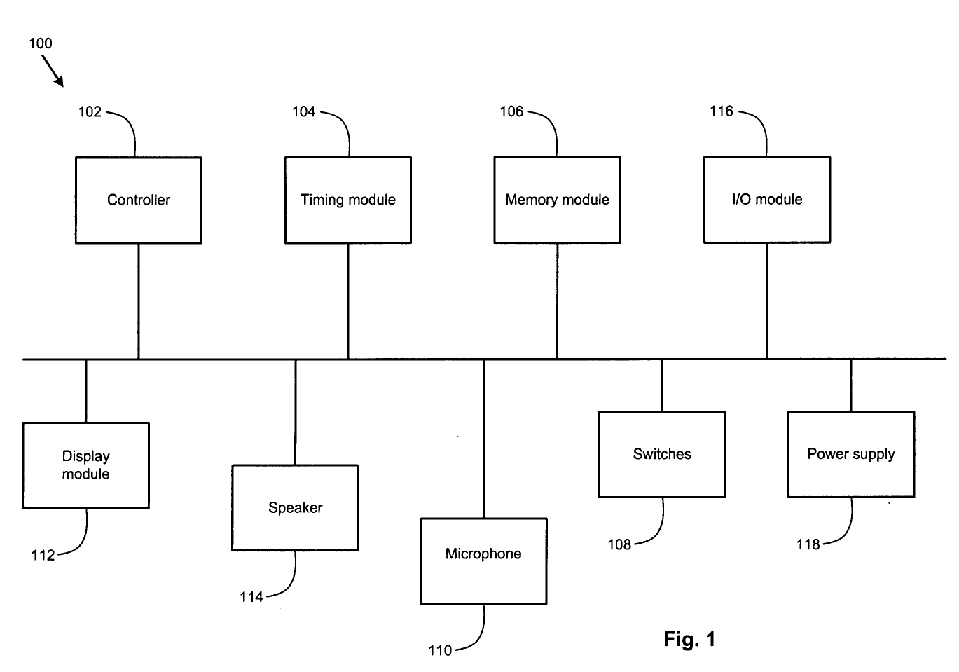

FIG. 1 is a basic system diagram of the assistive device 100 according to one embodiment. The purpose of this description is to introduce the basic components of the device. A detailed description of the functionality of the device will follow.

As shown in FIG. 1, the device includes a controller 102 that controls the various components of the device. The controller can be logic based, such as an application specific integrated circuit (ASIC), a field programmable gate array (FPGA), etc. One advantage of FPGA embodiments is that the controller logic can be updated. The controller can also be a microprocessor operating under a software-based operating system such as WINDOWS CE available from MICROSOFT Corporation. In a variation, the controller can be integrated into one of the other components of the device.

The device also includes a timing module 104 that maintains an internal clock and controls the alarm features that will hereafter be described in more detail.

A memory module 106 stores both system data and user input. Examples of data that may be stored in the memory module include user settings, voice recordings, software, etc. The memory in the memory module can include both random access memory (RAM) and read only memory (ROM). The memory can also include other types of storage media, such as optical media, tape, etc.

In one embodiment, the memory can include removable media, such as COMPACTFLASH® cards, Memory Stick cards, SD and miniSD cards, SmartMedia cards, etc. These removable memory media are available at commercial retail stores and also from SanDisk Corporation, 140 Caspian Court, Sunnyvale, Calif. 94089, USA. Other types of removable media include the aforementioned tapes and optical media (e.g., CD, DVD, CD±R, DVD±R, CD±RW, DVD±RW). The device would then, of course, include the appropriate reader port and logic to communicate with the appropriate memory media. Removable media is particularly useful because it allows the user to select the overall memory capacity of the device. Further, removable media allows a user to store data from another device, e.g., second management device, personal computer (PC), handheld computing device, tape recorder, etc. for use with the present device.

The device also includes various user input components. One component is a set of switches 108 that receive input from the user. The switches can include buttons, keys, latches, etc. Another component is a microphone 110 that receives audible input from the user. The user input components allow the user to provide instructions to the device, manage the settings of the device, record verbal messages, etc.

A display module 112 provides visual output to the user. The display module can display graphical data such as text and time information, animations, etc.

A speaker 114 provides audible output to the user, such as verbal recordings recorded via the microphone, alarm “beeps”, etc.

The device can also include a mechanism to provide vibration (not shown) upon occurrence of an event, such as upon execution of a prespecified alarm.

An in/out (I/O) module 116 allows the device to communicate with external devices.

A power supply 118 provides power to the device. The power supply can be commercially available disposable batteries. However, the power supply preferably includes one or more rechargeable batteries, which are charged by connecting the device to an external power supply or that can be charged in a charging unit. The device preferably also includes the capability of functioning directly from a constant power source, e.g., electrical plug.

Note that many of these modules can be implemented in hardware and/or software.

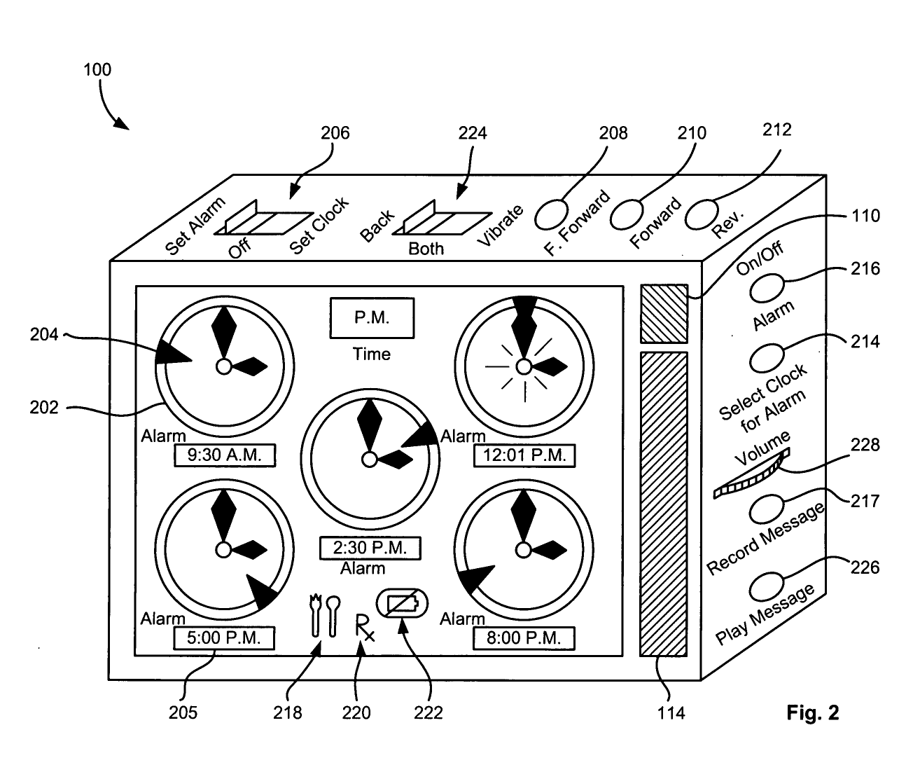

Use of the assistive device will now be presented in conjunction with the embodiment of the assistive device 100 shown in FIG. 2. As mentioned above, the timing of medication or food intake is important to effective control of some medical conditions. For instance, people with diabetes need to eat a certain amount of carbohydrates and take insulin at various times throughout the day. However, particularly for young children and older patients with poor memory, the capacity to remember and even know how much food or medicine to take, and when, can be very challenging.

The assistive device allows not only the ability to set reminder alarms, but also to record and play back audible reminders and instructions. The device further comprises a face which might comprise a glass-covered liquid crystal display as is known in the prior art, or may alternatively be one of a variety of other known display types. The face of the device includes several clock faces 202 of the 12 hour type. Each clock face displays a current time and further includes a marker 204 indicating an alarm setting. Alternatively, each clock face might show an alarm setting. In the embodiment shown, the device has five clock faces, though one skilled in the art will appreciate that more or less clock faces can be provided. Display of multiple clock faces are preferred so that the patient or caregiver can quickly see all set alarms.

Adjacent each clock face, there is preferably provided a digital time display 205 as is known in the prior art, which might, for example, include a small a.m/p.m. indicator so that a user or caregiver can quickly identify the exact time of the alarm.

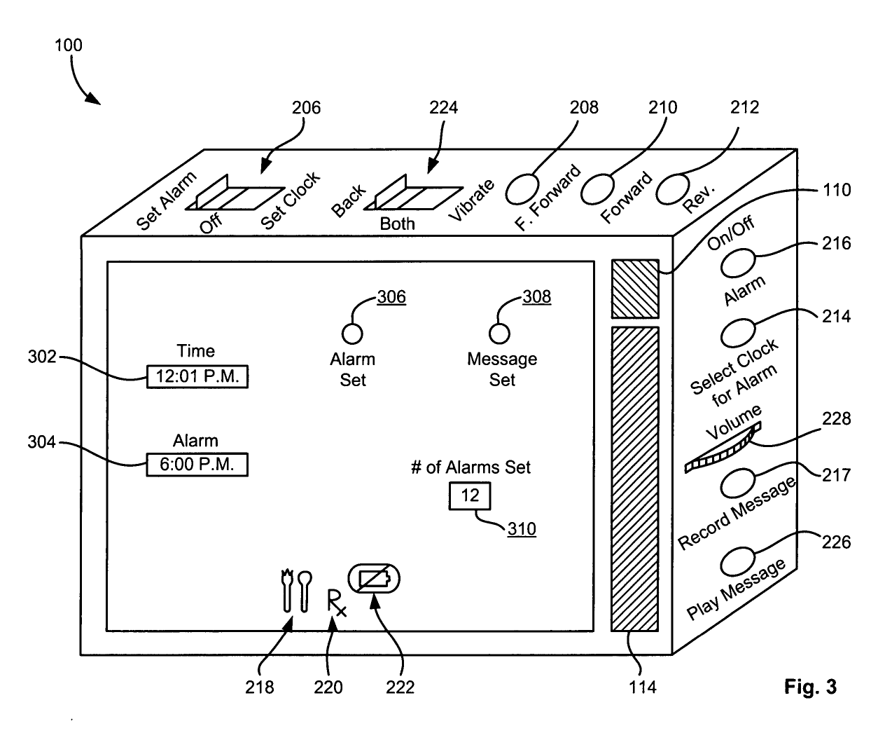

In a variation, a single clock face (digits or 12 hour type) can be displayed. The user could then scroll through various clock faces to see when additional alarms are set. Or, as shown in FIG. 3, two clock faces 302, 304 can be displayed, one for the current time and one for the presently selected/next alarm. The user can scroll through the alarm settings by actuating button 214. Indicators 306, 308 can illuminate when the displayed alarm is set and a message is set for the alarm. A number of alarms set can also be displayed in box 310.

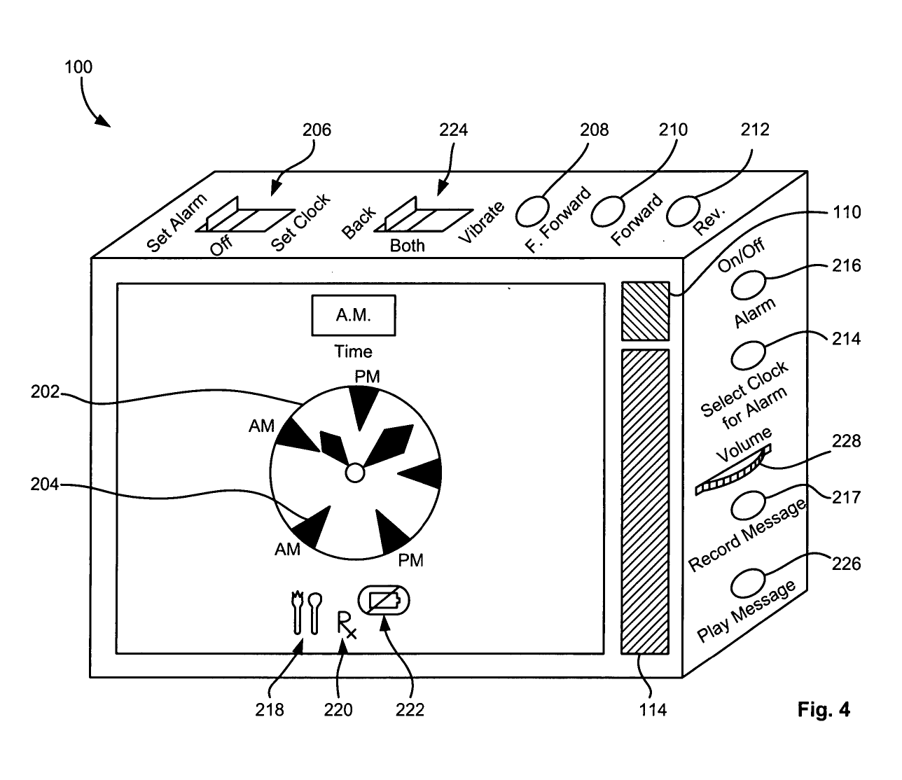

In a further variation, shown in FIG. 4, a single clock face can include markers for each alarm. Active alarms can be denoted by a marker having a different shape or color than alarms that are set but not active. Similarly, morning and evening alarms can have different shapes or colors to indicate am or pm.

In addition to the basic time and alarm functions, the device also includes keys (e.g., buttons) and/or switches for setting the time and alarm functions. Particularly, setting switch 206 to “set alarm” or “set clock” in conjunction with actuation of keys 208, 210, 212 allows the user to set the time or an alarm. Actuation of key 214 allows the user to select the clock for which an alarm is to be set. The active clock can be indicated by a change in color, change in lighting, flashing graphic, etc. Actuation of key 216 allows the user to turn an alarm on or off for the currently selected clock. A concealed switch (not shown) can also be provided for preventing accidental (or deliberate) erasure of one or more of the verbal message or alarms. The switch can be concealed behind a door, or can be small such that it is only actuatable with a tool, e.g., the tip of a ball point pen.

The keys and latches are preferably positioned along a peripheral sidewall of the device. By positioning these keys/latches about the device face, the difficulty of pressing a proper key is reduced, since the keys are adjacent, as opposed to the four of a regular calculator-type device keypad. This is most beneficial for the smaller children and elderly, though more convenient for all persons using the device. Each of the keys is preferably recessed to avoid inadvertent actuation, yet provide easy access for use.

Along with each alarm, a verbal message can be recorded and stored by selecting the record message button 217 while switch 206 is set to “set alarm”. In this way, a caregiver or user can record instructions as to the amount of food to eat or medicine to take at a certain time, the time corresponding to an alarm. For instance, assume a diabetic child needs to eat a certain amount of carbohydrates and take insulin at breakfast, 10 a.m., lunchtime, 3 p.m., dinnertime, and at bedtime. The parent can set an alarm to execute (go off) at each of these times, and record a verbal message for each alarm instructing the child how much insulin to take and/or carbohydrates to eat at each meal or snack. This feature is particularly useful for children and elderly who may not always be under the direct supervision of a caregiver.

By allowing the recording of verbal messages, the device can be tailored to each specific patient. Because conditions such as diabetes can change from day to day, the device provides an advantage over devices having only prestored graphical or textual messages.

By selecting a specific alarm, the user can review the message associated with that alarm. Preferably, each message is stored until recorded over. In addition, verbal messages can be recorded and stored in a library for later association with a particular alarm. This allows reuse of recorded messages for such things as football night, “hot dog Tuesday” at school, etc.

Several special icons can also be provided on the face, including a food icon 218 indicating time to eat, a medicine icon 220 indicating time to take medicine, and low battery icon 222. The low battery icon may alternatively be an icon signaling either device malfunction or impending malfunction, or such feature may be provided for separately. The icons illustrated are preferred, though one of ordinary skill in the art will understand that other suitable icons or indicators may be selected.

Though not specifically illustrated, the device is preferably assembled using known device assembly materials and techniques to be waterproof and durable. A back light is provided for easy viewing at night or in a dimly lit environment. An imprint may also be provided somewhere on the device which identifies the wearer as diabetic, such as on the back casing of the device. Over a period of time, the preferred embodiment will then be recognizable by the medical community as a medical identification.

A protective case can also be provided to house the device. The housing and/or protective case can include a clip or hook that attaches to, for example, a belt, pocket or bag. The device can also be worn as a wristwatch, necklace, etc.

The preferred device is provided through software with a total of eight alarm settings, though once again more or fewer settings may be provided. Eight is a comfortable number to accommodate most users, while keeping cost and complexity of the device to a minimum. These different settings may be for alarm function only, or a person may program one or more of the icons to appear with each alarm setting. This may be achieved by one of various techniques known in the art, such as pressing and holding alarm button for an appropriate amount of time while device cycles through each possible combination, pressing an icon selection button (not shown) multiple times to manually sequence through the various icons, or selection of one or more numeric buttons to activate one or more of the icons.

As is known in the art, various activities are desirably set to occur at particular times each day. By appropriate programming of the alarms, at the appropriate times the device may be used to signal a necessary activity. For example, a 6:30 a.m. alarm might be programmed to signal the need for a blood sugar test, an insulin injection, and potential time to eat. For this particular alarm, both icons 218 and 220 should be activated, and an accompanying audio alarm will chime. A silent alarm, e.g., vibration, may be used, as are known in the art of paging devices, though the chime is preferred. Selection of audible alarm, silent alarm, or both, is set by switch 224. If a verbal message has been recorded for that alarm, a light can flash, chime sound, or vibration occur to instruct the patient to listen to the recorded message. The user can then select the play message key 226 to replay the verbal message recorded for that particular alarm. A volume dial 228 allows the user to adjust the volume of the message and/or audio alarm. By requiring an additional step to output the message upon execution of the alarm, the privacy of the patient is assured as the patient can listen to the message at a preferred time and location. Note, though, that the verbal message can be output automatically upon execution of the alarm.

Additional functionality can be provided, such as synchronization with other devices via I/O module 116. For instance, one device can be programmed, and the settings and recordings stored therein can be sent to another device. One device is held by the patient, and the other by a caregiver or custodian, thereby providing redundant functionality and the corresponding safeguards. The device can also be connected to a computing device (e.g., PC, PDA, etc.), allowing the user to create and store settings on the computer which are then downloaded to the device. Data from the device can also be uploaded to the computing device. Additionally, the removable media described above can be used to transfer data between devices.

The device can also allow the patient to keep a verbal log of what was eaten, what the blood sugar readings were, and how much insulin was taken. The device can also add a time stamp to the recording, indicating the time that the recording is made. Preferably, a second “record” switch is provided for patient to record messages for the log. The second record switch can be of a different size, e.g., larger, and/or of a different color than the primary voice recording switch to facilitate identification and use by a child or elderly patient. When the second record switch is activated, the device creates a time stamp and records the voice of the user. When the second switch is activated again, the device repeats the process, storing a second message with time stamp. The verbal log function allows a caregiver to review recent events sequentially to e.g., ensure that the patient did what he was supposed to do at the proper time. This also would allow a doctor to diagnose the reason of a complication or attack.

A concealed switch similar to the one described above can be provided to access the patient's messages, as well as erase those messages.

Because the device is programmable, the user or a physician, dietician, parent or other concerned party is able to enter alarm times and record verbal messages to meet the user's needs. These needs can then be maintained as established by the responsible caregiver, physician or dietician. By combining the important features of time keeping and visual and verbal alarms in an easy-to-use device, the primary objectives of the present invention are met.

One skilled in the art will also appreciate that the salient features of the device can be incorporated into other devices such as pagers, cellular telephones, etc. without straying from the spirit and scope of the present invention. Also, the device can incorporate additional functionality such as a pager circuit, telephone circuit, etc. without straying from the spirit and scope of the present invention.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

What is claimed is:

1. A device for assisting in management of food and medicine intake, comprising:
a plurality of alarms each set to correspond to a time for a patient to intake at least one of food and medicine;
a display for displaying a time setting for each of the alarms;
a mechanism for storing verbal messages containing recorded human voice instructions for taking the at least one of the food and medicine; and
a mechanism for audibly outputting the verbal messages.

2. The device as recited in claim 1, further comprising a mechanism for recording the verbal messages and associating the recorded verbal messages with the alarms.

3. The device as recited in claim 2, wherein the verbal messages are stored in a library for later association with the alarms.

4. The device as recited in claim 1, wherein a user of the device must perform an additional step to hear the verbal messages upon execution of the alarms.

5. The device as recited in claim 1, wherein the display displays several clock faces simultaneously.

6. The device as recited in claim 5, wherein each of the clock faces indicates a time setting of the alarms.

7. The device as recited in claim 1, wherein the display displays a single clock face and markers for all alarms.

8. The device as recited in claim 1, wherein the patient has diabetes.

9. The device as recited in claim 8, wherein the verbal messages provide instructions relating to an amount of carbohydrates for the patient to eat.

10. The device as recited in claim 8, wherein the verbal messages provide instructions relating to an amount of insulin for the patient to take.

11. The device as recited in claim 1, wherein the patient is a child.

12. The device as recited in claim 1, wherein the patient is elderly.

13. The device as recited in claim 1, wherein the device synchronizes settings for the alarms with a second device having functionality similar to the device.

14. The device as recited in claim 13, wherein the device also transfers the verbal messages.

15. The device as recited in claim 1, wherein the device receives settings for the alarms from a second device.

16. The device as recited in claim 15, wherein the device receives the verbal messages from a second device.

17. The device as recited in claim 1, further comprising a mechanism for recording verbal input from the patient for keeping an audible log of events.

18. The device as recited in claim 1, further comprising a concealed switch for selectively preventing erasure of at least one of the verbal message or at least one of the alarms.

19. The device as recited in claim 1, further comprising a pager circuit.

20. The device as recited in claim 1, further comprising a telephone circuit.

21. A device for assisting in management of food and medicine intake for a diabetic person, comprising:
a plurality of alarms each set to correspond to a time for a diabetic patient to intake at least one of food and insulin;
a display for displaying a time setting for each of the alarms;
a mechanism for recording verbal messages containing recorded human voice instructions for taking the at least one of the food and insulin;
a mechanism for storing the verbal messages and associating the recorded verbal messages with the alarms; and
a mechanism for audibly outputting the verbal messages.

22. A method for assisting in management of food and medicine intake, comprising:
setting alarms to each correspond to a time for a patient to intake at least one of food and medicine;
displaying a time setting for each of the alarms;
storing verbal messages containing recorded human voice instructions for taking the at least one of the food and medicine; and
audibly outputting the verbal messages.
